(19) United States
(12) Patent Application Publication
(10) Pub. No.: US 2010/0087733 A1
(43) Pub. Date: Apr. 8, 2010

(54) BIOLOGICAL INFORMATION PROCESSING APPARATUS AND BIOLOGICAL INFORMATION PROCESSING METHOD

(75) Inventors: Takao Nakajima, Ebina-shi (JP); Kazuhiko Fukutani, Yokohama-shi (JP); Yasufumi Asao, Atsugi-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/574,086

(22) Filed: Oct. 6, 2009

(30) Foreign Application Priority Data
Oct. 7, 2008 (JP) 2008-260885

(51) Int. Cl. A61B 8/00; A61B 6/00

(52) U.S. Cl. 600/437; 600/476

(57) ABSTRACT

A biological information processing apparatus has a light source 102 which emits a light 101 to a living body 100; an acoustic wave detector 106 that detects an acoustic wave 105 which a light absorber 104 in the living body generates when it absorbs the light, and that converts the acoustic wave to an electric signal; and a signal processing unit 108 that obtains information in the living body from the electric signal and a light quantity distribution in the living body determined based on a shape of the living body.

### BACKGROUND OF THE INVENTION

1. Field of the Invention

The present invention relates to a biological information processing apparatus and a biological information processing method.

2. Description of the Related Art

In general, an imaging apparatus using an X-ray, an ultrasonic wave, and MRI (nuclear magnetic resonance) is often used in the medical field. An optical imaging apparatus which propagates a light emitted from a light source such as a laser beam into a specimen such as a living body, detects its propagated light, and obtains information in the living body is being studied actively in the medical field. As one of such optical imaging techniques, photoacoustic tomography (PAT) has been proposed (see Non Patent Literature (NPL) 1).

[NPL 1] M. Xu, L. V. Wang, “Photoacoustic imaging in biomedicine”, Review of scientific instruments, 77, 041101 (2006)

PAT is a technique that irradiates a specimen (living body) with a light of pulse emitted from a light source, detects an acoustic wave generated from a living tissue which absorbed the energy of the light propagated and diffused in the specimen at multiple positions, performs analytic processing of their signals, and visualizes information related to optical properties in the specimen. Accordingly, it is possible to obtain an optical property distribution, in particular, an optical energy absorption density distribution in the specimen.

According to NPL 1, in PAT, an initial sound pressure (P₀) of an optical acoustic wave generated from a light absorber in a specimen due to the light absorption is provided by the following expression.

P₀=Γ·μₐ·Φ (1)

Here Γ is a Grüneisen coefficient (parameter), which is obtained by dividing the product of a thermal expansivity (β) and the square of a speed of sound (c) by a constant pressure specific heat (C_{P}). μₐ is an optical absorption coefficient of the light absorber, and Φ is a light quantity in a local region (a quantity of light irradiated to the light absorber; also called “light fluence”). Since it is known that Γ will take an almost constant value if the tissue is decided, the product of μₐ and Φ in each local region, i.e., an optical energy absorption density distribution can be obtained by measuring and analyzing change of the sound pressure P, which is the magnitude of the acoustic waves, at multiple positions.

### SUMMARY OF THE INVENTION

In conventional PAT, as understood from Expression (1), in order to determine the optical absorption coefficient (μₐ) in the specific position in the specimen from the measured result of the sound pressure (P), the light quantity (Φ) emitted to the specific position need be determined. That is, in order to determine the optical absorption coefficient (μₐ) distribution in the living body, the optical energy absorption density distribution need be compensated for by the light quantity (Φ) distribution in the living body.

When it is assumed that the light quantity (Φ₀) emitted from the light source to the living body is constant and that the light is emitted to a large region with respect to the thickness of the living body so as to be propagated in the living body like a plane wave, the light quantity distribution (Φ) can be approximated by the following expression.

Φ=k·Φ₀·exp(−μ_{eff}·d₁) (2)

Here, μ_{eff} is an average effective attenuation coefficient of the living body, Φ₀ is a light quantity incident from the light source into the living body, and k is a coefficient depending on the optical absorption coefficient (μₐ) and the effective optical scattering coefficient (μₛ′). The “average” effective attenuation coefficient is referred to as the effective attenuation coefficient “when it is assumed that the optical property in the living body is spatially uniform”. In this specification, the “average” optical property means the optical property “when it is assumed that the optical property in the living body is spatially uniform”. Furthermore, d₁ is the distance from the region on the living body to which the light from the light source is emitted (light irradiated region) to the light absorber in the living body, that is, the depth of the light absorber.

In such model in which the light is attenuated in the living body in terms of an exponential function, the light quantity in the living body can be determined using the analytic solution, as Expression (2). This can perform light quantity compensation in a depth direction with respect to light irradiation. However, the light quantity distribution can be represented using such analytic solution only for a specific living body shape and a specific emitted light.

When the shape of the living body is not simple and the light irradiation distribution is not uniform, the light quantity distribution in the living body cannot be represented by such analytic solution model. Particularly, when light irradiation in a wide range is not uniform, the light quantity distribution is not uniform in an in-plane direction with respect to the irradiated surface in the living body. Accordingly, light quantity compensation in consideration of the non-uniformity is necessary.

In view of the above problems, an object of the present invention is to provide a technique for imaging an optical absorption coefficient (μₐ) distribution in a living body more accurately in photoacoustic tomography.

The present invention in its first aspect provides a biological information processing apparatus comprising: a light source which emits a light to a living body; an acoustic wave detector that detects an acoustic wave which a light absorber in the living body generates when the light absorber absorbs the light, and that converts the acoustic wave to an electric signal; and a signal processing unit that obtains information in the living body from the electric signal and a light quantity distribution in the living body determined based on a shape of the living body.

The present invention in its second aspect provides a biological information processing method comprising the steps of: detecting an acoustic wave which a light absorber in a living body generates when the light absorber absorbs a light emitted to the living body to convert the acoustic wave to an electric signal; and obtaining information in the living body from the electric signal and a light quantity distribution in the living body determined based on a shape of the living body.

According to the present invention, the optical absorption coefficient (μₐ) distribution in the living body can be obtained more accurately in photoacoustic tomography.

Further features of the present invention will become apparent from the following description of exemplary embodiments with reference to the attached drawings.

### BRIEF DESCRIPTION OF THE DRAWINGS

FIG. 1 is a diagram illustrating an example of the configuration of a biological information processing apparatus according to first and second embodiments of the present invention;

FIG. 2 is a diagram illustrating an example of the configuration of the biological information processing apparatus according to the first and second embodiments of the present invention;

FIG. 3 is a diagram illustrating an example of a process performed by the biological information processing apparatus according to the first embodiment of the present invention;

FIG. 4 is a flowchart illustrating an example of the process performed by the biological information processing apparatus according to the first embodiment of the present invention;

FIG. 5 is a flowchart illustrating an example of the process performed by the biological information processing apparatus according to the second embodiment of the present invention; and

FIG. 6 is a diagram illustrating an example of the configuration of the biological information processing apparatus according to a third embodiment of the present invention.

### DESCRIPTION OF THE EMBODIMENTS

Preferred embodiments of this invention will be illustratively described below in detail with reference to the drawings.

### First Embodiment

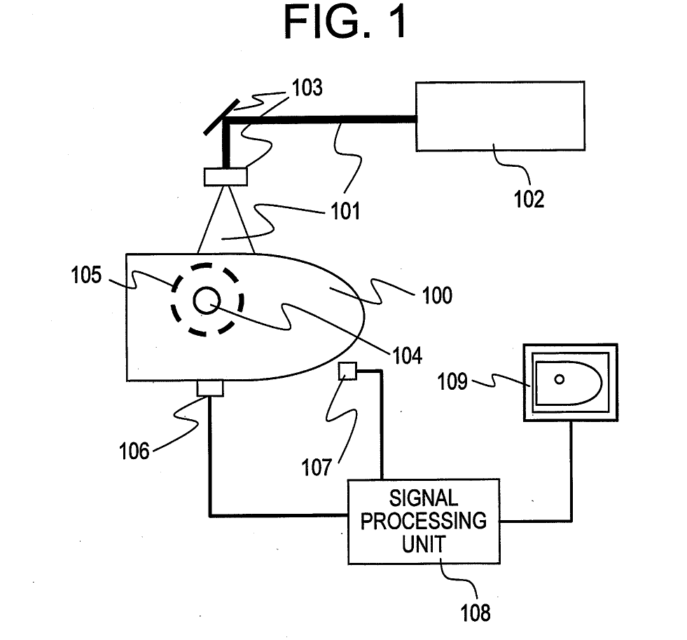

FIG. 1 illustrates the configuration of a biological information processing apparatus according to a first embodiment of the present invention. The first embodiment of the present invention will be described with reference to FIG. 1. The biological information processing apparatus described here can image the optical property distribution in a living body and the density distribution of a substance configuring a biological tissue obtained from the information for the diagnosis of a malignant tumor and a blood vessel disease and the progress observation of chemical treatment. That is, the biological information processing apparatus of the present invention preferably functions as a biological information imaging apparatus.

The biological information processing apparatus has a light source 102, an optical device 103, an acoustic wave detector (also called a probe) 106, a measuring unit 107, a signal processing unit 108, and a display unit 109. The light source 102 is a device which emits a light 101. The optical device 103 has an optical system configured of a lens, a mirror, and an optical fiber. The light 101 emitted from the light source 102 is guided by the optical device 103 and is then emitted to a living body 100. When, part of the energy of the light propagated in the living body 100 is absorbed to a light absorber 104 such as a blood vessel, an acoustic wave 105 is generated from the light absorber 104. In this specification, the “acoustic wave” is referred to as an elastic wave, typically an ultrasonic wave, generated by the photoacoustic effect from each of the local regions (light absorbers 104). The acoustic wave detector 106 is a device which detects the acoustic wave 105 generated from the light absorber 104 and converts the acoustic wave signal to an electric signal. The measuring unit 107 is a device which measures the shape of the living body 100 (at least the shape in the range in which the light 101 emitted from the light source 102 reaches the living body). The signal processing unit 108 determines the light quantity distribution in the living body based on the shape of the living body 100 measured by the measuring unit 107, stores the light quantity distribution, and obtains information (such as the optical property distribution) in the living body from the electric signal obtained from the acoustic wave detector 106 and the light quantity distribution. The display unit 109 is a device which displays image information obtained (reconstructed) by the signal processing unit 108. In the biological information processing apparatus of the present invention, the display unit 109 is not an essential component.

As described above, the acoustic wave is expressed by Expression (1). The Grüneisen coefficient (Γ) is almost constant if the tissue is found and is a known value. Accordingly, the initial sound pressure generation distribution or the product of the optical absorption coefficient (μₐ) and the light quantity (Φ) (the optical energy absorption density distribution) can be determined by the measurement and analysis of the change in the sound pressure (P) detected by the acoustic wave detector with time. The three-dimensional distribution of the light quantity (Φ) in the living body is derived based on the read shape of the living body 100. The optical energy absorption density distribution (μₐ·Φ) is compensated for by the light quantity distribution. Accordingly, the three-dimensional optical absorption coefficient (μₐ) distribution in the living body can be obtained.

In the image of the optical energy absorption density distribution, when the light absorbers having the same shape, size, and optical absorption coefficient exist indifferent positions in the living body, they are displayed with brightness or color different from each other. This is because the number of photons reaching each of the light absorbers, that is, the local light quantity in the living body, is different. On the other hand, when the light quantity distribution determined from the shape of the living body is used to perform light quantity compensation, as described above, the light absorbers having the same optical property (optical absorption coefficient) can be displayed with almost the same brightness or color in the finally obtained biological information image, which is advantageous for image diagnosis.

The configuration of the biological information processing apparatus of this embodiment will be described more specifically.

In FIG. 1, the light source 102 is a unit for irradiating light of a specific wave length that can be absorbed by a specific one of components of the living body. As the light source, there is provided at least one pulsed light source that can generate light of pulse (pulsed light) on the order of from several to hundreds nanoseconds. A laser is preferable as the light source, but it is also possible to use a light emitting diode or the like instead of the laser. As a laser, there can be used various kinds of laser such as a solid-state laser, a gas laser, a dye laser, a semiconductor laser, and so on. In addition, although in this embodiment, an example of a single light source is shown, a plurality of light sources can be used. In the case of the plurality of light sources, in order to raise the irradiation intensity of the light to be irradiated on the living body, there can be used two or more light sources which oscillate at the same wave length, or in order to measure differences in the optical property value distribution according to the wave lengths, two or more light sources having different oscillation wavelengths can be used. Here, note that if a dye of which the oscillating wave length is convertible, OPO (Optical Parametric Oscillators), or titanium-sapphire laser can be used as the light source, it will also become possible to measure the difference in the optical property value distributions depending upon the wave lengths. It is preferable that the wave lengths to be used be in a range of from 700 to 1,100 nm in which there is little absorption in the living body. However, in cases where an optical property value distribution of the living tissue relatively near a living body surface thereof, it is also possible to use a wave length range, such as for example a range of from 400 to 1,600 nm, wider than the above-mentioned wavelength range.

The light 101 emitted from the light source can be propagated using an optical waveguide. Although not illustrated in FIG. 1, an optical fiber is preferable as the optical waveguide. In case of using the optical fiber, respective optical fibers can be used for a plurality of light sources so as to guide the light to the surface of the living body, and all the lights from the a plurality of light sources may be guided to one optical fiber so as to be guided to the living body using only the one optical fiber. The optical device 103 has an optical component such as a mirror which reflects the light and a lens which focuses and enlarges the light and changes its shape. Any optical component which can emit the light 101 emitted from the light source 102 to the living body 100 may be used.

The biological information processing apparatus of this embodiment is intended for the diagnosis of a malignant tumor and a blood vessel disease of a human or an animal and the progress observation of chemical treatment. Accordingly, as the living body 100 as the specimen, the breasts, fingers, arms, and legs of a human or an animal to be diagnosed are assumed. Hemoglobin, a blood vessel including a large amount of hemoglobin, or a malignant tumor of a human body to be measured, which exhibits the high optical absorption coefficient in the specimen corresponds to the light absorber. Using a contrast agent introduced as the light absorber into the body, the biological information processing apparatus can be used for diagnosing a malignant tumor and a disease such as Alzheimer's disease and carotid artery plaque. As the contrast agent, for example, indocyanine green (ICG) and gold nanoparticles are used. However, any substance which generates the acoustic wave by light absorption may be used.

The acoustic wave detector (probe) 106 of FIG. 1 detects the acoustic wave (ultrasonic wave) 105 generated from the substance which absorbs part of the energy of the light 101 propagated in the living body and converts it to an electric signal. Any acoustic wave detector which can detect an acoustic wave signal, such as a transducer using a piezoelectric phenomenon, a transducer using light resonance, and a transducer using the change in capacitance, may be used. An arrayed transducer or a single-element transducer can be used. In this embodiment, to enable the detection of the acoustic waves 105 at a plurality of positions, one acoustic wave detector 106 is scanned on the surface of the living body 100. When the acoustic waves can be detected at the plurality of positions, the same effect can be obtained. Accordingly, a plurality of acoustic wave detectors may be arranged on the surface of the living body 100. An acoustic impedance matching agent, such as gel or water, for preventing the reflection of the acoustic wave is desirably used between the acoustic wave detector and the living body.

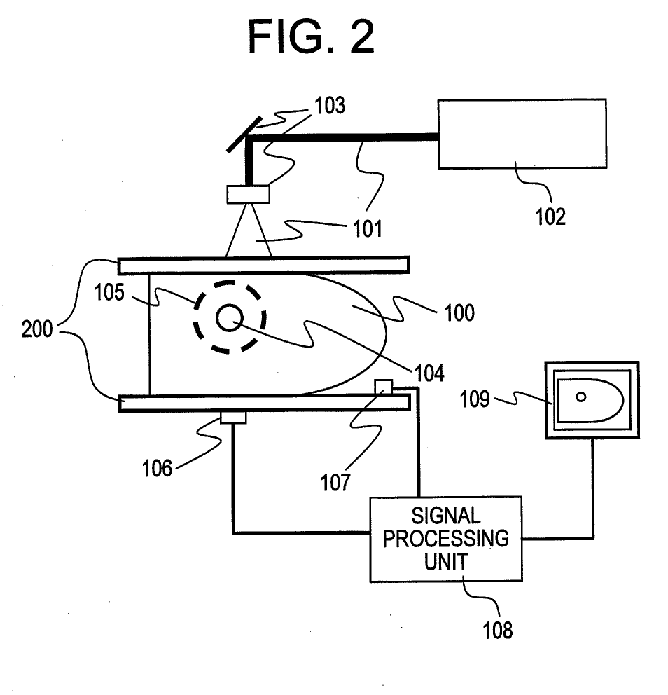

The measuring unit 107 is a device which measures the three-dimensional shape (e.g., thickness) of the living body 100. An imaging device such as a CCD camera can be used as the measuring unit 107. In the case, the signal processing unit calculates the outer shape or thickness of the living body from a fetched image. As illustrated in FIG. 2, when the biological information processing apparatus has fixing members 200 which fix (sandwich) the living body 100, a photometer which measures the thickness of the fixed living body (the distance between the two fixing members) can be used as the measuring unit 107. The measuring unit is not limited to such device, and any device which can measure the shape of the living body 100 may be used as the measuring unit 107. The shape or thickness of the living body may be measured by transmitting an ultrasonic wave from the acoustic wave detector 106 to perform echo measurement. In the case, the acoustic wave detector 106 serves as the measuring unit 107.

The signal processing unit 108 calculates the light quantity distribution in the living body based on the shape of the living body obtained by the measuring unit 107. As the calculation method of the light quantity distribution, the Monte Carlo method, the finite element method, or the like can be used. The present invention is not limited to such numerical calculation method. When the living body is fixed in a certain specific shape and is then irradiated under the specific light irradiation condition, e.g., spot irradiation or is irradiated with a uniform wide light in a wide range, the light quantity distribution can be calculated from the analytic solution. When the light quantity distribution is calculated, the shape of the living body and the optical coefficient (optical property) such as light absorption and optical scattering in the living body are necessary. In this embodiment, the predetermined average optical coefficient in the living body, that is, the average optical coefficient specific to the measured portion of the living body, is used for calculating the light quantity distribution.

As the preferred embodiment, the shape of the living body is measured by the measuring unit 107 and the light quantity distribution in the living body is determined based on the measured biological information. However, the essence of the present invention is that the optical absorption coefficient is calculated from the light quantity distribution in the living body determined based on the shape of the living body and the acoustic signal of PAT. Accordingly, it is not indispensable that the measuring unit 107 measures the living body. For example, information on the previously grasped shape of the living body may be input to the biological information processing apparatus of the present invention. The signal processing unit 108 may calculate the optical absorption coefficient using the light quantity distribution determined from the information. That is, it is essential only that the biological information processing apparatus of the present invention have means for obtaining information on the shape of the living body.

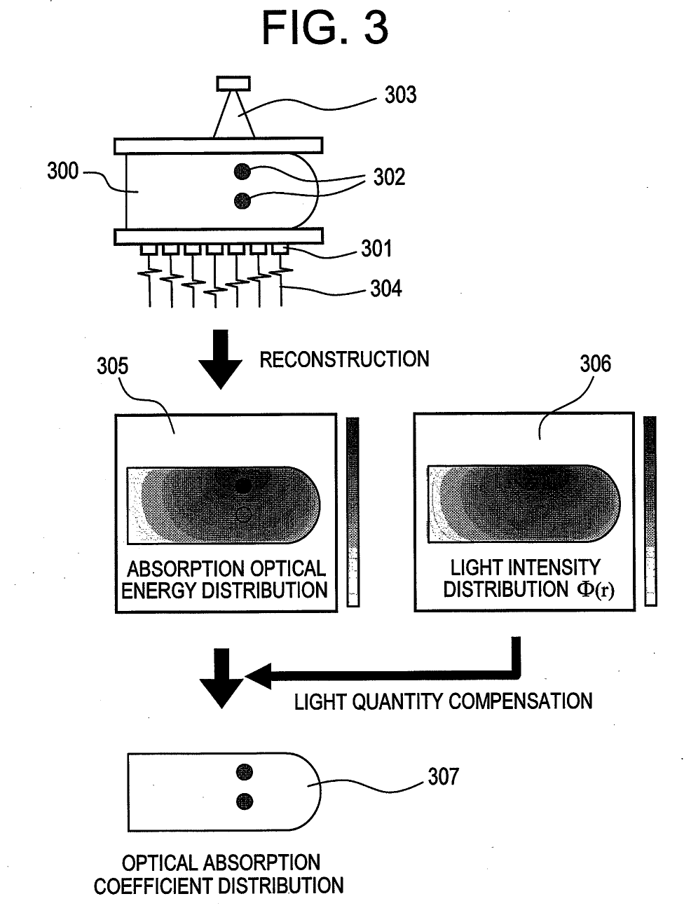

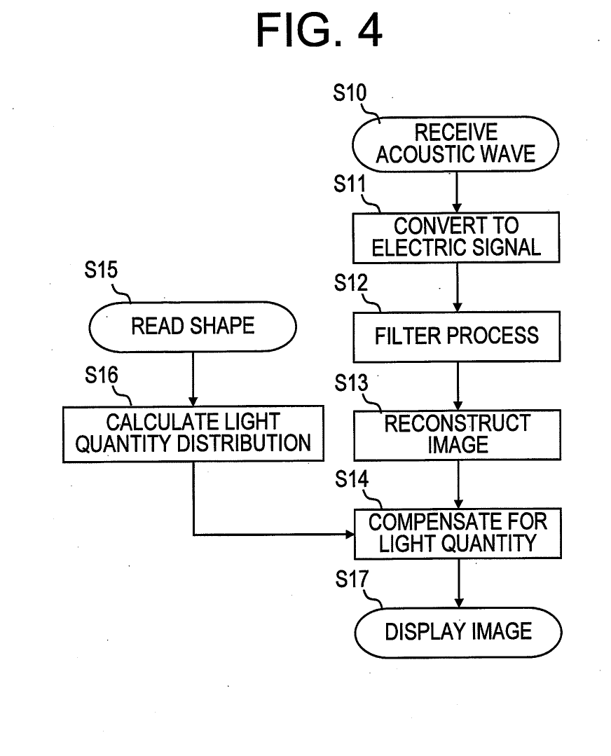

Referring to FIGS. 3 and 4, the operation of the biological information processing apparatus of this embodiment will be described.

A light of pulse (pulsed light) 303 is emitted from a light source to a living body 300 and an acoustic wave generated by a light absorber 302 in the living body is received by an acoustic wave detector 301 (S10). The acoustic wave signal is converted to an electric signal 304 by the acoustic wave detector 301 (S11) and is then fetched into the signal processing unit 108 (see FIGS. 1 and 2). The signal processing unit 108 subjects the electric signal 304 to the filter process and so on (S12), calculates the position and size of the light absorber 302 or an optical property distribution 305 such as the absorption optical energy distribution (the optical energy stacked amount distribution), and reconstructs an optical property distribution image (S13).

The signal processing unit 108 determines the shape (here, thickness) of the living body 300 from the information obtained by the measuring unit 107 (see FIGS. 1 and 2) (S15), and calculates a light quantity distribution (light intensity distribution) 306 in the living body based on its shape (S16).

The signal processing unit 108 uses the light quantity distribution calculated in S16 to perform light quantity compensation of the optical property distribution 305 obtained in S13 for determining an optical absorption coefficient distribution 307 (S14). Specifically, the optical energy stacked amount is expressed by the product of the optical absorption coefficient and the reached light quantity. Accordingly, the light quantity distribution can be compensated for by dividing the optical energy stacked amount distribution by the light quantity distribution. Thus, an image showing the obtained optical absorption coefficient distribution 307 is output to the display unit 109 (S17).

As described above, the signal processing unit 108 determines the initial sound pressure generation distribution or the product of the optical absorption coefficient (μₐ) and the light quantity (Φ) (the optical energy absorption density distribution) from the electric signal. The signal processing unit 108 calculates the light quantity distribution in the living body and performs light quantity compensation with respect to the product of the optical absorption coefficient (μₐ) and the light quantity (Φ) (the optical energy absorption density distribution). Accordingly, the optical absorption coefficient (μₐ) distribution in the specimen can be obtained.

As the signal processing unit 108, any device which can store the electric signal, convert it to data of the optical property distribution, store the shape of the living body, and calculate the light quantity distribution may be used. For example, the signal processing unit 108 can be configured of an oscilloscope and a computer analyzing the obtained data. As the display unit 109, any device which can display image data created by the signal processing unit 108 can be used. A liquid crystal display can be used.

When light of a plurality of wavelengths is used, the optical absorption coefficient distribution in the specimen is calculated with respect to the respective wavelengths, those values and the wavelength dependence specific to the substance configuring the biological tissue are compared, thereby imaging the density distribution of the substance configuring the living body. As the substance configuring the biological tissue, glucose, collagen, and oxidation-reduction hemoglobin are assumed.

According to the biological information processing apparatus of the above configuration, the optical property distribution in the living body, in particular, the optical absorption coefficient (μₐ) distribution, can be accurately imaged in photoacoustic tomography.

### Second Embodiment

Next, a second embodiment of the present invention will be described with reference to the drawings. The apparatus configuration is the same as the first embodiment in FIG. 1.

The signal processing unit 108 of this embodiment has a table (memory) which stores a plurality of previously-calculated pseudo light quantity distributions. The pseudo light quantity distribution is data showing the light quantity distribution in the living body and is previously calculated for various assumed shapes of the living body and optical coefficients. The Monte Carlo method, the finite element method, or the like can be used as the calculation method of the light quantity distribution. The present invention is not limited to such numerical value calculation method. The light quantity distribution can be calculated from the analytic solution, as described in the first embodiment. When the light quantity distribution is calculated, the shape of the living body and the optical coefficient (optical property) such as light absorption and optical scattering in the living body are necessary. In this embodiment, the predetermined average optical coefficient in the living body is used for calculating the light quantity distribution.

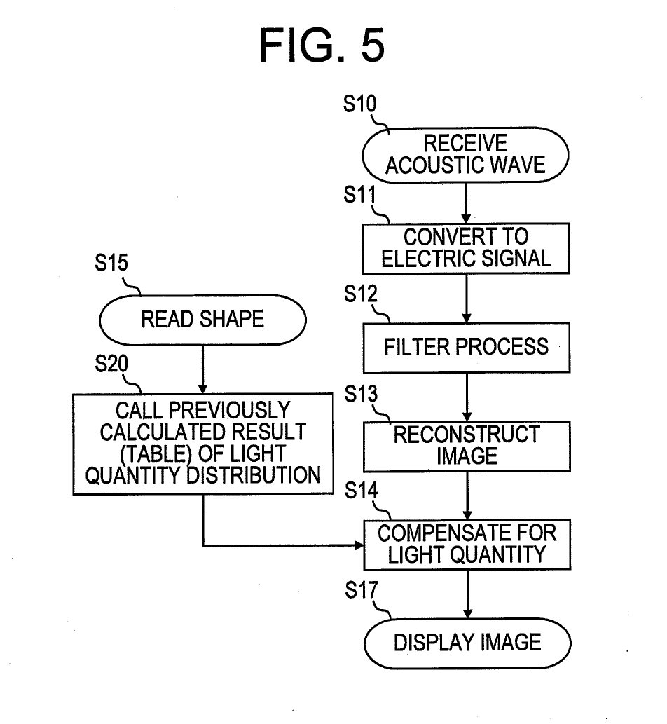

Referring to FIGS. 1 and 5, the operation of the biological information processing apparatus of this embodiment will be described.

The light of pulse 101 is emitted from the light source 102 to the living body 100 and the acoustic wave generated by the light absorber 104 in the living body is received by the acoustic wave detector 106 (S10). The acoustic wave signal is converted to an electric signal by the acoustic wave detector 106 (S11) and is then fetched into the signal processing unit 108. The signal processing unit 108 subjects the electric signal to the filter process and so on (S12), calculates the initial sound pressure generation distribution or the product of the optical absorption coefficient (μₐ) and the light quantity (Φ) (the optical energy absorption density distribution), and reconstructs an optical property distribution image (S13).

The signal processing unit 108 determines the shape of the living body 100 from the information obtained by the measuring unit 107 (S15), and selects the light quantity distribution corresponding to the shape of the living body from among the plurality of pseudo light quantity distributions in the table (S20).

The signal processing unit 108 uses the light quantity distribution determined in S20 to perform light quantity compensation of the optical property distribution obtained in S13, and can obtain the optical absorption coefficient distribution (μₐ) in the living body (S14). Thus, the image showing the obtained optical absorption coefficient distribution is output to the display unit 109 (S17).

As the signal processing unit 108, any device which can store the electric signal, convert it to data of the optical property distribution, and call data corresponding to the measured shape of the living body from the table which stores the pseudo light quantity distribution according to the shape of the living body may be used. For example, the signal processing unit 108 can be configured of an oscilloscope and a computer analyzing the obtained data.

As in the first embodiment, according to the biological information processing apparatus of this embodiment, the optical property distribution in the living body, in particular, the optical absorption coefficient (μₐ) distribution, can be imaged in photoacoustic tomography.

### Third Embodiment

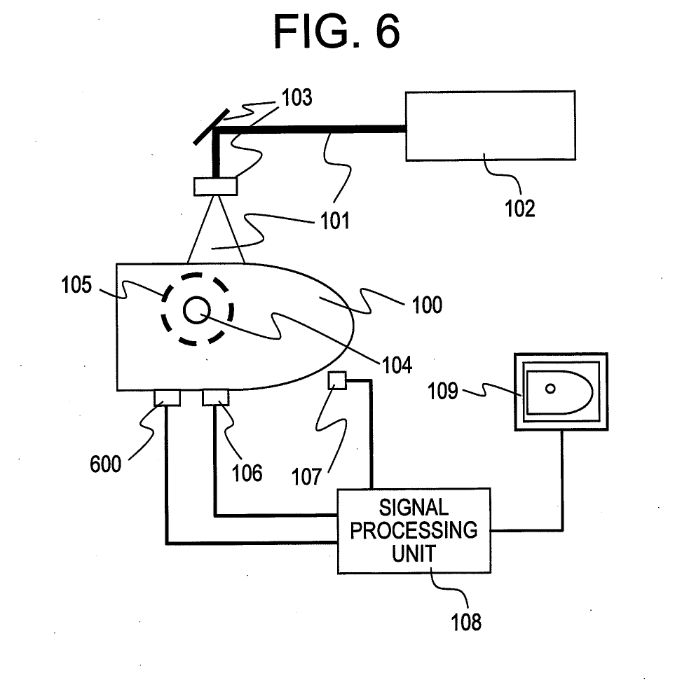

A third embodiment of the present invention will be described with reference to the drawings. The apparatus configuration is illustrated in FIG. 6.

The biological information processing apparatus of this embodiment has a second measuring unit which measures the average optical property (optical coefficient) in the living body. The signal processing unit 108 calculates the light quantity distribution using the optical measured value observed by the second measuring unit. The second measuring unit is configured of an optical detector 600 which detects the light propagated in the living body 100 and discharged to the outside of the living body.

As illustrated in FIG. 6, the light of pulse 101 is emitted from the light source 102 to the living body 100 and the acoustic wave generated by the light absorber 104 in the living body is received by the acoustic wave detector 106 so as to be converted to a first electric signal. The light propagated in the living body and discharged to the outside is detected by the optical detector 600 and is converted to a second electric signal. The shape of the living body is measured by the measuring unit 107.

The signal processing unit 108 determines the average optical coefficient in the living body by the second electric signal and determines the shape of the living body 100 from the information obtained by the measuring unit 107. The signal processing unit 108 uses the average optical coefficient obtained by the observation and the shape of the living body to calculate the light quantity distribution in the living body. When the table of the pseudo light quantity distribution is stored, the pseudo light quantity distribution corresponding to the optical coefficient and the shape of the living body may be called from the table.

The signal processing unit 108 subjects the first electric signal to the filter process and so on, and calculates the initial sound pressure generation distribution or the product of the optical absorption coefficient (μₐ) and the light quantity (Φ) (the optical energy absorption density distribution). The signal processing unit 108 uses the light quantity distribution in the living body to perform light quantity compensation with respect to the optical energy absorption density distribution, and can obtain the optical absorption coefficient distribution (μₐ) in the living body.

In this embodiment, when the first electric signal and the second electric signal are measured, the same light source 102 is used. However, different light sources may be used. That is, when the second electric signal is measured in the optical detector 600, a second light source different from the light source 102 can be used. The light emitted from the second light source to the living body 100 is not limited to the light of pulse. Either an intensity modulated light or a continuous wave light can be used.

In this embodiment, as illustrated in FIG. 2, the biological information processing apparatus can have the fixing members 200 which fix (sandwich) the living body 100.

According to the biological information processing apparatus of this embodiment, as in the first and second embodiments, the optical property distribution in the living body, in particular, the optical absorption coefficient (μₐ) distribution, can be imaged in photoacoustic tomography.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-260885, filed on Oct. 7, 2008, which is hereby incorporated by reference herein in its entirety.

What is claimed is:

1. A biological information processing apparatus comprising:
a light source which emits a light to a living body;
an acoustic wave detector that detects an acoustic wave which a light absorber in the living body generates when the light absorber absorbs the light, and that converts the acoustic wave to an electric signal; and
a signal processing unit that obtains information in the living body from the electric signal and a light quantity distribution in the living body determined based on a shape of the living body.

2. A biological information processing apparatus according to claim 1, further comprising:
a measuring unit that measures the shape of the living body,
wherein the light quantity distribution is determined based on the shape of the living body measured by the measuring unit.

3. A biological information processing apparatus according to claim 1,
wherein the light quantity distribution is a three-dimensional light quantity distribution in the living body.

4. A biological information processing apparatus according to claim 1,
wherein the obtained information in the living body is an optical absorption coefficient distribution in the living body.

5. A biological information processing apparatus according to claim 1,
wherein the signal processing unit calculates the light quantity distribution using a predetermined average optical property in the living body.

6. A biological information processing apparatus according to claim 1, further comprising:
a second measuring unit which measures a average optical property in the living body,
wherein the signal processing unit calculates the light quantity distribution using the average optical property in the living body measured by the second measuring unit.

7. A biological information processing apparatus according to claim 6,
wherein the second measuring unit is an optical detector which detects a light propagated in the living body and discharged to the outside of the living body.

8. A biological information processing apparatus according to claim 1,
wherein the signal processing unit stores a plurality of pseudo light quantity distributions previously calculated corresponding to each of a plurality of shapes and selects the light quantity distribution corresponding to the measured shape of the living body from among the plurality of pseudo light quantity distributions.

9. A biological information processing apparatus according to claim 2,
wherein the measuring unit is a device which measures a thickness of the living body.

10. A biological information processing apparatus according to claim 1,
wherein the light source is a light source which generates a light of pulse.

11. A biological information processing apparatus according to claim 1,
wherein the acoustic wave detector can detect the acoustic waves at a plurality of positions.

12. A biological information processing apparatus according to claim 1,
wherein a wavelength of the light is in the range from 400 nm to 1,600 nm.

13. A biological information processing apparatus according to claim 1,
wherein the light absorber is a contrast agent introduced into the living body.

14. A biological information processing method comprising the steps of:
detecting an acoustic wave which a light absorber in a living body generates when the light absorber absorbs a light emitted to the living body to convert the acoustic wave to an electric signal; and
obtaining information in the living body from the electric signal and a light quantity distribution in the living body determined based on a shape of the living body.

15. A biological information processing method according to claim 14,
further comprising a step of measuring the shape of the living body,
wherein the light quantity distribution is determined based on the measured shape of the living body.
